(19) United States
(12) Patent Application Publication
(10) Pub. No.: US 2007/0016985 A1
(43) Pub. Date: Jan. 18, 2007

(54) Particle Preparation for Direct-Delivery Transformation

(75) Inventors: Michael D. Miller, Winterset, IA (US); Gary A. Sandahl, West Des Moines, IA (US); David J. Peterson, Ames, IA (US); Bruce J. Drummond, Johnston, IA (US); Mark A. Chamberlin, Windsor Heights, IA (US); Benjamin A. Bowen, Berkeley, CA (US); Grace M. St. Clair, Des Moines, IA (US); William J. Gordon-Kamm, Urbandale, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: Pioneer Hi-Bred International, Inc., Johnston, IA (US)

(21) Appl. No.: 11/458,219

(22) Filed: Jul. 18, 2006

Related U.S. Application Data
(60) Provisional application No. 60/700,225, filed on Jul. 18, 2005.
(60) Provisional application No. 60/801,004, filed on May 17, 2006.

(51) Int. Cl. A01H 11/00; A01H 9/00

(52) U.S. Cl. 800/295

(57) ABSTRACT

Methods of preparing microparticles for transformation of plant cells with compositions of interest, and compositions comprising the prepared particles and the associated compound(s) of interest are provided. Further provided are methods to deliver compositions of interest to plant cells for transient or stable incorporation in the genome, and any transformed plant cells, plants, and seeds produced thereby.

### RELATED APPLICATIONS

This application claims priority to U.S. Ser. No. 60/700,225 filed Jul. 18, 2005, and U.S. Ser. No. 60/801,004 filed May. 17, 2006, the disclosures of which are herein incorporated in their entirety by reference.

### TECHNICAL FIELD

The present invention relates to the preparation of microparticles for the transformation of plants.

### BACKGROUND

Plants have been transformed using a variety of methods, including bombardment of plant cells with dense microparticles carrying molecules of interest such as polynucleotides. Biolistic transformation methods can be used with essentially any plant species that can be cultured in vitro for stable and/or transient transformation, where Agrobacterium-mediated methods may have a more limited scope of target plants and/or tissues and are not typically used for transient transformation. Typically, polynucleotides are bound to the microparticles by precipitation of DNA using a chemical such as calcium chloride, and/or spermidine. This method may not be compatible for delivery of certain polynucleotides or other compositions such as polypeptides, subcellular organelles, microorganisms, or any combinations of these. A continuing need exists for methods to deliver a variety of compositions to plant tissues for transformation.

### SUMMARY

Methods of preparing microparticles for transformation of plant cells with compositions of interest, and compositions comprising the prepared particles and the associated compound(s) of interest are provided. Further provided are methods to deliver compositions of interest to plant cells for transient or stable incorporation in the genome, and any transformed plant cells, plants, and seeds produced thereby.

### DETAILED DESCRIPTION

Various compounds can be used to prepare microparticles for particle-mediated direct delivery methods to introduce compositions into plant cells. For example, microprojectiles for a particle gun method, or whisker/needles can be prepared by associating the composition of interest to be delivered with the microprojectiles in the presence of an association agent including but not limited to a polyelectrolytes, polyampholytes, fatty acids, neutral lipid, cationic lipid solution, liposome solution, cationic polymer, DNA binding protein, cationic protein, cationic peptide, polyamino acids, surfactants, detergents, or any combination(s) thereof. In some examples the compound is a cationic lipid solution comprising N,N,N′,N′-tetramethyl-N,N′-bis(2-hydroxylethyl)-2,3-di(oleoyloxy)-1,4-butanediammonium iodide. In some examples the cationic lipid solution further comprises L-dioleoyl phosphatidylethanolamine (DOPE). In some examples, the particles for direct delivery are prepared by associating the composition of interest with the microprojectiles in the presence of Tfx-10™, Tfx-20™, Tfx-50™, Lipofectin™, Lipofectamine™, Cellfectin™, Effectene™, Cytofectin GSV™, Perfect Lipids™ DOTAP, DMRIE-C, FuGENE-6™, Superfect™, Polyfect™, polyethyleneimine (PEI), chitosan, protamine CI, DNA binding proteins, histone H1, histone CENH3, poly-L lysine, DMSA, and the like.

Compositions comprising the microparticles, associated composition of interest, and the association agent are provided. Microparticles include any solid carrier used for delivery of a composition of interest into the interior of a cell. Any microparticle can be used, examples of microparticles include but are not limited to metal particles such as gold and tungsten particles used for particle bombardment of cells, and gold nanoparticles (Au NPs) used in cellular uptake; whiskers such as alumina, silica, glass, ceramic, titania, zirconia, boron, carbon, carbides, silicon carbide whiskers; carbon nanofibers, optionally arrayed as vertically aligned carbon nanofiber (VACNFs) arrays; and nanomaterials such as mesoporous silicate nanoparticles (MSN), and the like. Microparticles used for particle bombardment are typically made from metals such as gold or tungsten, and range in size from about 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, or 2.0 μm. Microparticles for use for whiskers-mediated transformation are typically made of silicon carbide, and range in length from about 4 μm, 5 μm, 6 μm, 7 μm, 8 μm, 9 μm, 10 μm, 12 μm, 15 μm, 20 μm, 25 μm 30 μm, or 40 μm, and range in width from 0.2 μm, 0.3 μm, 0.4 μm, 0.5 μm, 0.6 μm, 0.7 μm, 0.8 μm, 0.9 μm, or 1.0 μm with commonly used whiskers including, for example 30 μm×0.5 μm and 10 μm×0.3 μm. In some examples the composition of interest is more uniformly deposited on the microparticle as compared to a standard control. In some examples the microparticles comprising the composition of interest forms a more uniform suspension as compared to a standard control. In some examples the composition of interest is a polynucleotide composition, and the standard control particle preparation comprises a CaCl₂-spermidine precipitation.

In some examples the composition of interest to be delivered comprises a polynucleotide composition. Polynucleotides are any nucleic acid molecule polymer, and comprise naturally occurring, synthetic, and/or modified ribonucleotides, deoxyribonucleotides, and combinations of ribonucleotides and deoxyribonucleotides. Polynucleotides encompass all forms of sequences including, but not limited to, single-stranded, double-stranded, triplexes, linear, circular, branched, hairpins, stem-loop structures, branched structures, and the like. Polynucleotides include native DNA, native RNA, genomic fragments, synthesized molecules, cloned fragments, and any combination thereof. The polynucleotides can be any size from short oligonucleotides typically less than about 120 nucleotides in length, small polynucleotides typically about 120 nucleotides to about 2 kb, moderately sized polynucleotides typically greater than 2 kb to about 20 kb, large polynucleotides typically greater than about 20 kb to about 75 kb, and very large polynucleotides greater than 75 kb. Polynucleotides include native DNA, native RNA, genomic fragments, synthesized molecules, cloned fragments, and any combination thereof. A polynucleotide composition comprises at least one polynucleotide species, and includes mixtures of polynucleotide species wherein each polynucleotide species in the mixture each have at least one distinct characteristic as compared to any other member of the mixture such as size, sequence composition, strandedness, physical form, modified bases, and the like. In some examples the polynucleotide is a DNA construct generated using any molecular biological technique or synthetic technique to juxtapose at least two heterologous nucleic acid sequences in a polynucleotide. Unless otherwise stated a polynucleotide composition comprises at least one polynucleotide species wherein the polynucleotide species is greater than 120 nucleotides in length. If the polynucleotide composition comprises a mixture of polynucleotide species, at least one polynucleotide species in the mixture is greater than 120 nucleotides in length. In some examples the polynucleotide composition comprises at least one polynucleotide that encodes a polypeptide that can enhance or stimulate cell growth, a recombinase, an integrase, a site-specific recombinase, a homing meganuclease, a transposase, a meganuclease, a restriction enzyme, a transcription factor, a repressor, a screenable marker, and/or a zinc-finger protein. In some examples the composition of interest is more uniformly deposited on the microparticle as compared to a standard control CaCl₂-spermidine precipitation. In some examples the microparticles comprising the composition of interest forms a more uniform suspension as compared to a CaCl₂-spermidine precipitation standard control.

In some examples the composition of interest to be delivered comprises a polypeptide composition. Polypeptides are any amino acid polymer, and comprise naturally occurring, synthetic, and/or modified amino acids in any physical confirmation including linear, circular, branched, secondary, tertiary, and quaternary structures, and any combination thereof. A polypeptide composition comprises at least one polypeptide species, and includes mixtures of polypeptide species wherein each polypeptide species in the mixture each have at least one distinct characteristic as compared to any other member of the mixture such as size, sequence composition, physical form, modified amino acid, and the like. In some examples the polypeptide composition comprises at least one polypeptide that can enhance or stimulate cell growth, a recombinase, an integrase, a site-specific recombinase, a homing meganuclease, a transposase, a meganuclease, a restriction enzyme, a transcription factor, a repressor, a screenable marker, and/or a zinc-finger protein.

In some examples the composition of interest to be delivered comprises a microorganism. In some examples the microorganism is a virus or a bacterial cell, and the target cell is a eukaryotic cell. In some examples the bacterial cell is an Agrobacterium, and the eukaryotic cell is a plant cell. For example an Agrobacterium comprising a T-DNA containing a polynucleotide of interest can be associated with the microparticle. In some examples the microorganism deposited on the microparticle has an improved viability as compared to another method. In some examples the microorganism deposited on the microparticle has an improved viability as compared to another control method, wherein the microorganism is Agrobacterium and the control method comprises drying Agrobacterium cell suspension in growth media onto the microparticles. The microparticle can be used to deliver the Agrobacterium and its T-DNA to a plant cell, wherein the T-DNA can transfer the polynucleotide of interest to the plant cell. In some examples the polynucleotide of interest is stably incorporated into a genome of a plant cell.

In some examples the composition of interest to be delivered comprises a subcellular organelle composition. In some examples the subcellular organelle composition is a plastid, such as a chloroplast or a mitochondrion, or a nucleus for example to provide a means for in vitro fertilization of a eukaryotic cell.

In some example the composition of interest comprises a polynucleotide composition and a polypeptide composition. In some examples the polypeptide composition comprises a polypeptide that can enhance or stimulate cell growth, a recombinase, an integrase, a site-specific recombinase, a homing meganuclease, a transposase, a meganuclease, a restriction enzyme, a transcription factor, a repressor, a screenable marker, and/or a zinc-finger protein. In some examples the polynucleotide composition comprises at least one polynucleotide of interest to be stably incorporated into a genome of a plant cell. In some examples the composition of interest comprises a protein-polynucleotide complex. In some examples the protein in the protein-polynucleotide complex is a DNA binding protein, including but not limited to a recombinase, a transcription factor, a DNA repair protein, a repressor, a transactivating factor, a zinc-finger protein, a leucine-zipper protein, a cell cycle protein, a meganuclease, a DNA polymerase, a DNA ligase, and the like. In some examples the protein in the protein-polynucleotide complex is a RNA binding protein, including but not limited to a DICER, a DICER-LIKE protein, a Drosha, a Rnase, a RNA-dependent RNA polymerase, ribosomal proteins, and the like. In some examples the composition of interest is more uniformly deposited on the microparticle as compared to a standard control CaCl₂-spermidine precipitation. In some examples the microparticles comprising the composition of interest forms a more uniform suspension as compared to a CaCl₂-spermidine precipitation standard control.

In some examples, upon delivery into the cell at least one component of the composition of interest dissociates from the microparticle. In some examples the composition of interest is a polynucleotide composition. In some examples the polynucleotide composition comprises or encodes a RNA of interest which is expressed in the cell. In some examples the polynucleotide composition encodes a polypeptide of interest which is expressed in the cell. In some examples the expression of the RNA and/or polypeptide is transient. In some examples the polynucleotide composition dissociates from the microparticle, and at least one polynucleotide component is stably integrated in a genome of the cell to produce a transformed cell. In some examples the polynucleotide composition encodes a polypeptide that can enhance or stimulate cell growth, a recombinase, an integrase, a site-specific recombinase, a homing meganuclease, a transposase, a meganuclease, a restriction enzyme, a transcription factor, a repressor, a screenable marker, and/or a zinc-finger protein. In some examples the polynucleotide composition comprises a polynucleotide that can suppress the expression of a target molecule in the cell. In some examples the polynucleotide composition comprises a double-stranded RNA, miRNA precursor, a miRNA, a siRNA precursor, a siRNA, a transacting siRNA precursor, a transacting siRNA, an RNAi precursor, an antisense polynucleotide precursor, an antisense polynucleotide, a sense-suppression precursor, a sense-suppression polynucleotide, or a ribozyme. In some examples the composition of interest is a polypeptide composition. In some examples the polynucleotide composition comprises a minichromosome polynucleotide. A minichromosome polynucleotide encompasses satellite minichromosomes, artificial chromosomes, supernumerary chromosomes, chromosome fragments, and the like that are stably transmitted to a daughter cell during mitosis, wherein the minichromosome comprises euchromatin, heterochromatin, or any combination of euchromatin and heterochromatin. In some examples the polynucleotide composition comprises a mixture of minichromosome polynucleotides. In some examples the minichromosome polynucleotide comprises a DNA construct. In some examples the minichromosome polynucleotide comprises a genomic fragment. In some examples the minichromosome polynucleotide comprises a BAC clone comprising a maize centromeric repeat, a telomere, and/or a origin of replication functional in a plant.

In some examples, upon delivery into the cell at least one component of the composition of interest does not dissociate from the microparticle, but remains bound to the microparticle, for example providing a means for transient delivery or expression of a polynucleotide or polypeptide of interest. In some examples the association agent used comprises PEI. In some examples the composition of interest is a polynucleotide composition. In some examples the polynucleotide composition is more uniformly deposited on the microparticle as compared to a standard control CaCl₂-spermidine precipitation. In some examples the microparticles comprising the polynucleotide composition forms a more uniform suspension as compared to a CaCl₂-spermidine precipitation standard control. In some examples the polynucleotide composition comprises or encodes a RNA of interest which is expressed in the cell. In some examples the polynucleotide composition encodes a polypeptide of interest which is expressed in the cell. In some examples the polynucleotide composition encodes a polypeptide that can enhance or stimulate cell growth. In some examples the polynucleotide composition comprises a polynucleotide that can suppress the expression of a target molecule in the cell. In some examples the polynucleotide composition comprises a miRNA precursor, a miRNA, a siRNA precursor, a siRNA, a transacting siRNA precursor, a transacting siRNA, an RNAi precursor, an antisense polynucleotide precursor, an antisense polynucleotide, a sense-suppression precursor, a sense-suppression polynucleotide, or a ribozyme. In some examples the composition of interest is a polypeptide composition. In some examples the polypeptide composition comprises a polypeptide that can enhance or stimulate cell growth, a recombinase, an integrase, a site-specific recombinase, a homing meganuclease, a transposase, a meganuclease, a restriction enzyme, a transcription factor, a repressor, a screenable marker, and/or a zinc-finger protein.

Methods to prepare microparticles to deliver a composition of interest to a cell are provided. In some examples the microparticles are prepared by providing microparticles suitable for direct-delivery into plant cells, providing the composition of interest, and contacting the composition of interest with the microparticles in the presence of a compound to produce a microparticle having the composition of interest attached thereto, wherein the compound is selected from the group consisting of polyelectrolytes, polyampholytes, fatty acids, neutral lipid, cationic lipid solution, liposome solution, cationic polymer, DNA binding protein, cationic protein, cationic peptide, polyamino acids, surfactants, detergents, or any combination(s) thereof. In some examples the compound is a cationic lipid solution comprising N,N,N′,N′-tetramethyl-N,N′-bis(2-hydroxylethyl)-2,3-di(oleoyloxy)-1,4-butanediammonium iodide. In some examples the cationic lipid solution further comprises L-dioleoyl phosphatidylethanolamine (DOPE). In some examples, the particles for direct delivery are prepared by associating the composition of interest with the microprojectiles in the presence of Tfx-1™, Tfx-20™, Tfx-50™, Lipofectin™, Lipofectamine™, Cellfectin™, Effectene™, Cytofectin GSV™, Perfect Lipids™ DOTAP, DMRIE-C, FuGENE-6™, Superfect™, Polyfect™, polyethyleneimine (PEI), chitosan, protamine CI, DNA binding proteins, histone H1, histone CENH3, poly-L lysine, DMSA, and the like. Any microparticles suitable for delivery of a composition of interest into a cell can be used. In some examples the microparticles are the particles used for particle bombardment transformation methods, such as gold (Au) particles, and tungsten particles, or the particles used for whiskers transformation methods such as silicon carbide whisker particles. In some examples the composition of interest comprises a composition selected from the group consisting of a polynucleotide composition, a polypeptide composition, a subcellular organelle composition, and a microorganism composition. In some examples the composition is a polynucleotide composition comprising at least one polynucleotide encoding a polypeptide that enhances or stimulates cell growth, a recombinase, an integrase, a site-specific recombinase, a homing meganuclease, a transposase, a meganuclease, a restriction enzyme, a transcription factor, a repressor, a screenable marker, and/or a zinc-finger protein. In some examples the polypeptide that enhances or stimulates cell growth is selected from the group consisting of a wuschel polypeptide and a babyboom polypeptide. In some examples the polynucleotide composition comprises a mixture of polynucleotide species. In some examples the polynucleotide composition comprises at least one polynucleotide greater than 20 kb in size. In some examples the polynucleotide composition comprises a BAC clone, or a polynucleotide derived from a BAC clone. In some example the polynucleotide composition comprises at least one polynucleotide comprising a maize centromeric region. Microparticles produced by the methods having the composition of interest attached thereto are also provided. In some examples the composition of interest is more uniformly deposited on the microparticle as compared to a standard control. In some examples the microparticles comprising the composition of interest forms a more uniform suspension as compared to a standard control. In some examples the composition of interest comprises a polynucleotide composition and is more uniformly deposited on the microparticle as compared to a standard control CaCl₂-spermidine precipitation. In some examples the microparticles comprising a polynucleotide composition forms a more uniform suspension as compared to a CaCl₂-spermidine precipitation standard control. In some examples the frequency of delivery of the composition of interest is increased as compared to a standard control. In some examples the frequency of delivery of a polynucleotide composition is increased as compared to a CaCl₂-spermidine precipitation standard control.

Methods to provide a composition of interest into plant cells are provided. In some examples the composition of interest is provided to plant cells by methods comprising providing microparticles suitable for direct-delivery into plant cells, providing the composition of interest, contacting the composition of interest with the microparticles in the presence of a compound to produce microparticles having the composition of interest attached thereto, wherein the compound is selected from the group consisting of a polyelectrolytes, polyampholytes, fatty acids, neutral lipid, cationic lipid solution, a liposome solution, a DNA binding protein, a cationic protein, a cationic peptide, a cationic polymer, polyamino acids, surfactants, detergents, and a cationic polyamino acid, and, contacting the microparticles produced in step (c) with plant cells such that the microparticles deliver the composition of interest to the interior of the plant cells. In some examples contacting the microparticles with the plant cells comprises particle bombardment. In some examples contacting the microparticles with the plant cells comprises mixing the microparticles with the cells, for example a whiskers-mediated transformation. In some examples contacting the microparticles with the cells comprises providing the microparticles to the cell culture under conditions wherein the cells take up the microparticles directly. In some examples the compound is a cationic lipid solution comprising N,N,N′,N′-tetramethyl-N,N′-bis(2-hydroxylethyl)-2,3-di(oleoyloxy)-1,4-butanediammonium iodide. In some examples the cationic lipid solution further comprises L-dioleoyl phosphatidylethanolamine (DOPE). In some examples, the particles for direct delivery are prepared by associating the composition of interest with the microprojectiles in the presence of Tfx-10™, Tfx-20™, Tfx-50™, Lipofectin™, Lipofectamine™, Cellfectin™, Effectene™, Cytofectin GSV™, Perfect Lipids™ DOTAP, DMRIE-C, FuGENE-6™, Superfect™, Polyfect™, polyethyleneimine (PEI), chitosan, protamine CI, DNA binding proteins, histone H1, histone CENH3, poly-L lysine, DMSA, and the like. Any microparticles suitable for delivery of a composition of interest into a cell can be used. In some examples the microparticles are the particles used for particle bombardment transformation methods, such as gold (Au) particles, and tungsten particles, or the particles used for whiskers transformation methods such as silicon carbide whisker particles. In some examples the composition of interest comprises a composition selected from the group consisting of a polynucleotide composition, a polypeptide composition, a subcellular organelle composition, and a microorganism composition. In some examples the composition is a polynucleotide composition comprising at least one polynucleotide encoding a polypeptide that enhances or stimulates cell growth, a site-specific recombinase, a homing meganuclease, a transposase, a meganuclease, a restriction enzyme, a transcription factor, a repressor, a screenable marker, and/or a zinc-finger protein. In some examples the polypeptide that enhances or stimulates cell growth is selected from the group consisting of a wuschel polypeptide and a babyboom polypeptide. In some examples the polynucleotide composition comprises a mixture of polynucleotide species. In some examples the polynucleotide composition comprises at least one polynucleotide greater than 20 kb in size. In some examples the polynucleotide composition comprises a BAC clone, or a polynucleotide derived from a BAC clone. In some example the polynucleotide composition comprises at least one polynucleotide comprising a maize centromeric region. While any plant cell can be used in some examples the plant cell is from a monocotyledonous or a dicotyledonous plant. In some examples the plant cell is selected from the group consisting of maize, rice, wheat, barley, millet, sorghum, rye, soybean, alfalfa, canola, Arabidopsis, tobacco, sunflower, cotton, and safflower. In some examples after delivery of the microparticles into plant cells the composition of interest does not substantially dissociate from the microparticles. In some examples the composition of interest comprises a polynucleotide composition comprising at least one polynucleotide of interest, wherein after delivery of the microparticles the polynucleotide of interest stably incorporates into a genome of a plant cell. In some examples polynucleotide of interest incorporates into the genome by means of a site-specific recombinase mediated recombination event. In some examples the composition of interest comprises a polynucleotide composition comprising at least one polynucleotide of interest, wherein after delivery of the microparticles the polynucleotide of interest does not stably incorporate into a genome of a plant cell. In some examples after delivery of the microparticles into plant cells the polynucleotide of interest is transiently expressed in the plant cells. Plant cells, plants, and seeds produced by the method and comprising the composition of interest are provided. In some examples the composition of interest is more uniformly deposited on the microparticle as compared to a standard control. In some examples the microparticles comprising the composition of interest forms a more uniform suspension as compared to a standard control. In some examples the composition of interest comprises a polynucleotide composition and is more uniformly deposited on the microparticle as compared to a standard control CaCl₂-spermidine precipitation. In some examples the microparticles comprising a polynucleotide composition forms a more uniform suspension as compared to a CaCl₂-spermidine precipitation standard control. In some examples the frequency of delivery of the composition of interest is increased as compared to a standard control. In some examples the frequency of delivery of a polynucleotide composition is increased as compared to a CaCl₂-spermidine precipitation standard control.

Any compound that will attach or associate the composition of interest to the microparticles can be used. In some examples the compound is a polyelectrolyte, polyampholyte, fatty acid, neutral lipid, lipid solutions, cationic lipid solution, a liposome solution, a ionic polymer, a anionic polymer, a cationic polymer, a protein, a DNA binding protein, a cationic protein, a cationic peptide, surfactant, detergent, polyamino acid, or a cationic polyamino acid. Polyelectrolytes are polymers whose repeating units bear an electrolyte group. These groups will dissociate in aqueous solutions, making the polymers charged. Polyelectrolyte properties are similar to both electrolytes (salts) and polymers. Many biological molecules are polyelectrolytes, for example polypeptides and polynucleotides DNA, and synthetic polyelectrolytes are widely available. Polyelectrolytes which bear both cationic and anionic repeat groups are called polyampholytes. A fatty acid is a carboxylic acid or organic acid, often with a long aliphatic tail, and includes saturated and unsaturated molecules. Fatty acids typically include chains as short as butyric acid (4 carbons). Fatty acids derived from natural fats and oils typically have at least 8 carbon atoms, e.g. caprylic acid (octanoic acid). Fatty acids can be synthesized by the hydrolysis of the ester linkages in a triglycerides, with the removal of glycerol. Cationic lipids have a net positive charge, and many are available for transfection of mammalian cells with polynucleotides and/or polypeptides. The transfection solution sometimes has a second lipid component, such as a neutral or fusogenic lipid, to facilitate uptake across the cell membrane. Many cationic lipids are commercially available including: 293fectin™ which comprises a proprietary cationic lipid based formula optimized for use with 293 cells; Cellfectin® comprising a cationic lipid 1:1.5 (M/M) liposome formulation of cationic lipid N, NI, NII, NIII-Tetramethyl-N,NI,NII,NIII-tetrapalmityl-spermine (TM-TPS), and dioleoyl phosphatidylethanolamine (DOPE); DMRIE-C comprising a cationic lipid 1:1 (M/M) liposome formulation of cationic lipid DMRIE 1,2-dimyristyloxy-propyl-3-dimethyl-hydroxy ethyl ammonium bromide and cholesterol; Freestyle™ MAX comprising a proprietary cationic lipid designed for Freestyle™ cells; Lipofectamine™ comprising a 3:1 (w/w) liposome formulation of polycationic lipid 2,3-dioleyloxy-N-[2(sperminecarboxamido)ethyl]-N,N-dimethyl-1-propanaminium trifluoroacetate (DOSPA) and neutral lipid dioleoyl phosphatidylethanolamine (DOPE) (all of which are available from InVitrogen); Tfx™ reagents (TFX-10, TFX-20, TFX-50) all contain the same concentration of the cationic lipid component with different molar ratios of the fusogenic lipid, comprising a mixture of a synthetic, cationic lipid molecule [N,N,N′,N′-tetramethyl-N,N′-bis(2-hydroxyethyl)-2,3-di(oleoyloxy)-1,4-butanediammonium iodide] and L-dioleoyl phosphatidylethanolamine (DOPE); TransFast™ comprising the synthetic cationic lipid, (+)-N,N[bis (2-hydroxyethyl)]-N-methyl-N-[2,3-di(tetradecanoyloxy)propyl]ammonium iodide and the neutral lipid, DOPE; Transfectam® comprises a synthetic, cationic lipopolyamine molecule dioctadecylamidoglycyl spermine (DOGS) with the spermine group is covalently attached through a peptide bond to the lipid moiety (all available from Promega); CLONfectin™ comprises a cationic, amphiphilic lipid that promotes the efficient delivery of plasmid DNA into mammalian cells via liposome-mediated transfection (available from CloneTech); ESCORT™ liposome transfection reagent comprises 1:1 (w/w) liposome formulation of the cationic lipid N-[1-(2,3-dioleoyloxy)propyl]-N,N,N-trimethylammonium chloride (DOTAP) and dioleoyl phosphatidylethanolamine (DOPE) in 0.2 μm filtered MES-buffered saline, pH 6.2; ESCORT II is a unique formulation of the neutral lipid dioleoyl phosphatidylethanolamine (DOPE) and a proprietary cationic lipid; and DOTAP methosulfate comprising N-(2,3-Dioleoyloxy-1-propyl)trimethylammonium methyl sulfate cationic liposome-forming compound (all available from Sigma Chemical Co.). Other compounds include hexadimethrine bromide 1,5-Dimethyl-1,5-diazaundecamethylene polymethobromide (Kawai S et al. (1984) Mol Cell Biol 4:1172-1174); poly-L-ornithine/DMSO; polybrene/DMSO; polybrene/glycerol; polyethyleneimine (PEI), chitosan, protamine CI, DNA binding proteins, histone H1, histone CENH3, poly-L lysine, DMSA, and the like.

In some examples genes and/or encoded polypeptides that can enhance or stimulate cell growth are provided. Genes that enhance or stimulate cell growth include genes involved in transcriptional regulation, homeotic gene regulation, stem cell maintenance and proliferation, cell division, and/or cell differentiation such as WUS homologues (Mayer et al. (1998) Cell 95:805-815; WO01/0023575; US2004/0166563); aintegumenta (ANT) (Klucher et al. (1996) Plant Cell 8:137-153; Elliott et al. (1996) Plant Cell 8:155-168; GenBank Accession Nos. U40256, U41339, Z47554); clavata (e.g., CLV1, CVL2, CLV3) (WO03/093450; Clark et al. (1997) Cell 89:575-585; Jeong et al. (1999) Plant Cell 11:1925-1934; Fletcher et al. (1999) Science 283:1911-1914); Clavata and Embryo Surround region genes (e.g., CLE) (Sharma et al. (2003) Plant Mol Biol 51:415-425; Hobe et al. (2003) Dev Genes Evol 213:371-381; Cock & McCormick (2001) Plant Physiol 126:939-942; Casamitjana-Martinez et al. (2003) Curr Biol 13:1435-1441); baby boom (e.g., BNM3, BBM, ODP1, ODP2) (WO00/75530; Boutileir et al. (2002) Plant Cell 14:1737-1749); Zwille (Lynn et al. (1999) Dev 126:469-481); leafy cotyledon (e.g., Lec1, Lec2) (Lotan et al. (1998) Cell 93:1195-1205; WO00/28058; Stone et al. (2001) Proc Natl Acad Sci USA 98:11806-11811; U.S. Pat. No. 6,492,577); Shoot Meristem-less (STM) (Long et al. (1996) Nature 379:66-69); ultrapetala (ULT) (Fletcher (2001) Dev 128:1323-1333); mitogen activated protein kinase (MAPK) (Jonak et al. (2002) Curr Opin Plant Biol 5:415); kinase associated protein phosphatase (KAPP) (Williams et al. (1997) Proc Natl Acad Sci USA 94:10467-10472; Trotochaud et al. (1999) Plant Cell 11:393-406); ROP GTPase (Wu et al. (2001) Plant Cell 13:2841-2856; Trotochaud et al. (1999) Plant Cell 11:393-406); fasciata (e.g. FAS1, FAS2) (Kaya et al. (2001) Cell 104:131-142); cell cycle genes (U.S. Pat. No. 6,518,487; WO99/61619; WO02/074909), Shepherd (SHD) (Ishiguro et al. (2002) EMBO J. 21:898-908); Poltergeist (Yu et al. (2000) Dev 127:1661-1670; Yu et al. (2003) Curr Biol 13:179-188); Pickle (PKL) (Ogas et al. (1999) Proc Natl Acad Sci USA 96:13839-13844); knox genes (e.g., KN1, KNAT1) (Jackson et al. (1994) Dev 120:405-413; Lincoln et al. (1994) Plant Cell 6:1859-1876; Venglat et al. (2002) Proc Natl Acad Sci USA 99:4730-4735); fertilization independent endosperm (FIE) (Ohad et al. (1999) Plant Cell 11:407-415), and the like. Single species or combinations of polynucleotides can be provided. The combinations include multiple copies of any one of the polynucleotides of interest, and may have any combination of up-regulating and down-regulating expression of the combined polynucleotides. The combinations may or may not be combined on one polynucleotide and therefore may be provided sequentially or simultaneously.

Elements from recombination systems, such as recombinases, and recombination sites can be provided, for example in a DNA construct, a target site, and/or a transfer cassette. A target site comprises a polynucleotide integrated into the genome, the polynucleotide comprising a promoter operably linked to at least one recombination site. A transfer cassette comprises at least a first recombination site operably linked to a polynucleotide of interest and/or a polynucleotide encoding a selection marker, wherein the first recombination site is recombinogenic with a recombination site in the target site. A targeted seed or plant has stably incorporated into its genome a DNA construct that has been generated and/or manipulated through the use of a recombination system. Site-specific recombination methods that result in various integration, alteration, and/or excision events to generate the recited DNA construct can be employed to generate a targeted seed. See, e.g., WO99/25821, WO99/25854, WO99/25840, WO99/25855, WO99/25853, WO99/23202, WO99/55851, WO01/07572, WO02/08409, and WO03/08045. Various components, including those from a site-specific recombination system, can be provided to a plant using a variety of transient methods. Such transient transformation methods include, but are not limited to, the introduction of the recombinase or active fragment or variant thereof directly, introduction of the recombinase mRNA, or using a non-integrative method, or introducing low levels of DNA into the plant. Such methods include, for example, microinjection, particle bombardment, viral vector systems, and/or precipitation of the polynucleotide wherein transcription occurs from the particle-bound DNA without substantive release from the particle or integration into the genome, such methods generally use particles coated with polyethylimine, (see, e.g., Crossway et al. (1986) Mol Gen Genet 202:179-185; Nomura et al. (1986) Plant Sci 44:53-58; Hepler et al. (1994) Proc Natl Acad Sci USA 91:2176-2180; and Hush et al. (1994) J Cell Sci 107:775-784).

Any site-specific recombination system or component of thereof may be used. A site-specific recombinase, also referred to as a recombinase, is a polypeptide that catalyzes conservative site-specific recombination between its compatible recombination sites, and includes wild type sequences as well as a wide variety of modified sites that retain activity. A site-specific recombination site, or recombination site, is a polynucleotide sequence recognized by a site-specific recombinase as a substrate for the site-specific recombination reaction, and includes wild type sequences as well as a wide variety of modified sites that retain activity. For reviews of site-specific recombinases, see Sauer (1994) Curr Op Biotechnol 5:521-527; and Sadowski (1993) FASEB 7:760-767. Recombinases include recombinases from the integrase and the resolvase families. The integrase family of recombinases has over one hundred members and includes, for example, FLP, Cre, lambda integrase, and R. For other members of the integrase family, see for example, Esposito et al. (1997) Nucleic Acids Res 25:3605-3614 and Abremski et al. (1992) Protein Eng 5:87-91. Other recombination systems include, for example, the streptomycete bacteriophage phi C31 (Kuhstoss et al. (1991) J Mol Biol 20:897-908); the SSV1 site-specific recombination system from Sulfolobus shibatae (Maskhelishvili et al. (1993) Mol Gen Genet 237:334-342); and a retroviral integrase-based integration system (Tanaka et al. (1998) Gene 17:67-76). FLP recombinase catalyzes a site-specific reaction that is involved in amplifying the copy number of the two-micron plasmid of S. cerevisiae during DNA replication. FLP recombinase catalyzes site-specific recombination between two FRT sites. The FLP protein has been cloned and expressed (Cox (1993) Proc Natl Acad Sci USA 80:4223-4227). The FLP recombinase may be derived from the genus Saccharomyces. One can also synthesize a polynucleotide comprising the recombinase using plant-preferred codons for enhanced expression in a plant of interest. A recombinant FLP enzyme encoded by a nucleotide sequence comprising maize preferred codons (FLPm) that catalyzes site-specific recombination events is known (U.S. Pat. No. 5,929,301). Additional functional variants and fragments of FLP are known (Buchholz et al. (1998) Nat Biotechnol 16:617-618, Hartung et al. (1998) J Biol Chem 273:22884-22891, Saxena et al. (1997) Biochim Biophys Acta 1340:187-204, and Hartley et al. (1980) Nature 286:860-864). The bacteriophage recombinase Cre catalyzes site-specific recombination between two lox sites. The Cre recombinase is known (Guo et al. (1997) Nature 389:40-46; Abremski et al. (1984) J Biol Chem 259:1509-1514; Chen et al. (1996) Somat Cell Mol Genet 22:477-488; Shaikh et al. (1977) J Biol Chem 272:5695-5702; and, Buchholz et al. (1998) Nat Biotechnol 16:617-618. Cre polynucleotide sequences may also be synthesized using plant-preferred codons (e.g., moCre, WO99/25840). A chimeric recombinase can be also used, for example as described in WO99/25840.

A marker provides for the identification and/or selection of a cell, plant, and/or seed expressing the marker. Markers include, e.g., screenable, visual, and/or selectable marker. A selection marker is any marker, which when expressed at a sufficient level, confers resistance to a selective agent. For example visual markers can be used to identify transformed cells comprising the introduced DNA construct(s), in one example, the visual marker is a fluorescent protein. Such fluorescent proteins include but are not limited to yellow fluorescent protein (YFP), green fluorescent protein (GFP), cyan fluorescent protein (CFP), and red fluorescent protein (RFP). In still other examples, the visual marker is encoded by a polynucleotide having maize preferred codons. In further examples, the visual marker comprises GFPm, AmCyan, ZsYellow, or DsRed. See, Wenck et al. (2003) Plant Cell Rep 22:244-251. Selection markers and their corresponding selective agents include, but are not limited to, herbicide resistance genes and herbicides; antibiotic resistance genes and antibiotics; and other chemical resistance genes with their corresponding chemical agents. Bacterial drug resistance genes include, but are not limited to, neomycin phosphotransferase II (nptII) which confers resistance to kanamycin, paromycin, neomycin, and G418, and hygromycin phosphotransferase (hph) which confers resistance to hygromycin B. See also, Bowen (1993) Markers for Plant Gene Transfer, Transgenic Plants, Vol. 1, Engineering and Utilization; Everett et al. (1987) Bio/Technology 5:1201-1204; Bidney et al. (1992) Plant Mol Biol 18:301-313; and WO97/05829. Resistance may also be conferred to herbicides from several groups, including amino acid synthesis inhibitors, photosynthesis inhibitors, lipid inhibitors, growth regulators, cell membrane disrupters, pigment inhibitors, seedling growth inhibitors, including but not limited to imidazolinones, sulfonylureas, triazolopyrimidines, glyphosate, sethoxydim, fenoxaprop, glufosinate, phosphinothricin, triazines, bromoxynil, and the like. See, for example, Holt (1993) Ann Rev Plant Physiol Plant Mol Biol 44:203-229; and Miki et al. (2004) J Biotechnol 107:193-232. Selection markers include sequences that confer resistance to herbicides, including but not limited to, the bar gene, which encodes phosphinothricin acetyl transferase (PAT) which confers resistance to glufosinate (Thompson et al. (1987) EMBO J 6:2519-2523); glyphosate oxidoreductase (GOX), glyphosate N-acetyltransferase (GAT), and 5-enol pyruvylshikimate-3-phosphate synthase (EPSPS) which confer resistance to glyphosate (Barry et al. (1992) in Biosynthesis and Molecular Regulation of Amino Acids in Plants, B. K. Singh et al. (Eds) pp. 139-145; Kishore et al. (1992) Weed Tech 6:626-634; Castle (2004) Science 304:1151-1154; Zhou et al. (1995) Plant Cell Rep 15:159-163; WO97/04103; WO02/36782; and WO03/092360). Other selection markers include dihydrofolate reductase (DHFR), which confers resistance to methotrexate (see, e.g., Dhir et al. (1994) Improvements of Cereal Quality by Genetic Engineering, R. J. Henry (ed), Plenum Press, New York; and Hauptmann et al. (1988) Plant Physiol 86:602-606). Acetohydroxy acid synthase (AHAS or ALS) mutant sequences lead to resistance to imidiazolinones and/or sulfonylureas such as imazethapyr and/or chlorsulfuron (see, e.g., Zu et al. (2000) Nat Biotechnol 18:555-558; U.S. Pat. Nos. 6,444,875, and 6,660,910; Sathasivan et al. (1991) Plant Physiol 97:1044-1050; Ott et al. (1996) J Mol Biol 263:359-368; and Fang et al. (1992) Plant Mol Biol 18:1185-1187). In addition, chemical resistance genes further include tryptophan decarboxylase which confers resistance to 4-methyl tryptophan (4-mT) (Goodijn et al. (1993) Plant Mol Biol 22:907-912); and bromoxynil nitrilase which confers resistance to bromoxynil. The selection marker may comprise cyanamide hydratase (Cah), see, for example, Greiner et al. (1991) Proc Natl Acad Sci USA 88:4260-4264; and Weeks et al. (2000) Crop Sci 40:1749-1754. Cyanamide hydratase enzyme converts cyanamide into urea, thereby conferring resistance to cyanamide. Any form or derivative of cyanamide can be used as a selection agent including, but not limited to, calcium cyanamide (Perlka® (SKW, Trotberg Germany) and hydrogen cyanamide (Dormex® (SKW)). See also, U.S. Pat. Nos. 6,096,947, and 6,268,547.

In some examples the composition of interest comprises a polynucleotide composition encoding a repressor, a polypeptide composition comprising a repressor, and/or a microorganism comprising a repressor. Any repressor/operator system of interest can be used, including the Tet and the Lac repressor systems and any derivatives thereof. In some examples the composition of interest can comprise tetracycline-inducible and/or tetracycline-repressible promoters (Gatz et al. (1991) Mol Gen Genet 227:229-237; U.S. Pat. Nos. 5,814,618, and 5,789,156).

In some examples a polynucleotide composition comprises at least one DNA construct. A DNA construct comprises a polynucleotide which when present in the genome of a plant is heterologous or foreign to that chromosomal location in the plant genome. In preparing the DNA construct, various fragments may be manipulated to provide the sequences in a proper orientation and/or in the proper reading frame. Adapters or linkers may be employed to join the fragments. Other manipulations may be used to provide convenient restriction sites, removal of superfluous DNA, or removal of restriction sites. For example, in vitro mutagenesis, primer repair, restriction, annealing, resubstitutions, transitions, transversions, or recombination systems may be used. Polynucleotides of interest refer to any nucleic acid molecule included in the DNA construct(s) for any purpose, including but not limited to untranslated regions, regulatory regions, transcription initiation regions, translation initiation regions, introns, exons, polynucleotides encoding an RNA, selection markers, screenable markers, phenotypic markers, polynucleotides encoding a recombinase, recombination sites, target sites, transfer cassettes, restriction sites, recognition sites, insulators, enhancers, spacer/stuffer sequences, origins of replication, telomeric sequence, operators, and the like, can be provided in a DNA construct(s). The construct can include 5′ and 3′ regulatory sequences operably linked to the appropriate sequences. The DNA construct(s) can include in the 5′ to 3′ direction of transcription at least one of the following, a transcriptional and translational initiation region, the polynucleotide, and a transcriptional and translational termination region functional in plants. Alternatively, the DNA construct(s) may lack at least one 5′ and/or 3′ regulatory element. In some examples DNA construct(s) are designed such that upon introduction into a cell and in the presence of the appropriate recombinase a recombination event at the target site operably links the 5′ and/or 3′ regulatory regions to the appropriate sequences of the DNA construct(s). Operably linked means that the nucleic acid sequences linked are contiguous and comprise a functional linkage of the components. In some examples intervening sequences can be present between operably linked elements and not disrupt the functional linkage. For example, an operable linkage between a promoter and a polynucleotide of interest allows the promoter to initiate and mediate transcription of the polynucleotide of interest. In some examples a translational start site is operably linked to a recombination site. In some examples, a recombination site is within an intron. The cassette may additionally contain at least one additional sequence to be introduced into the plant. Alternatively, additional sequence(s) can be provided separately. DNA constructs can be provided with a plurality of restriction sites or recombination sites for manipulation of the various components and elements. DNA constructs may additionally contain selectable marker genes. Where appropriate, polynucleotides may be modified for increased expression in the transformed plant. For example, the polynucleotides can be synthesized using plant-preferred codons for improved expression. see, e.g., Campbell & Gowri (1990) Plant Physiol 92:1-11 for a discussion of host-preferred codon usage. Methods for synthesizing plant-preferred genes include, for example, U.S. Pat. Nos. 5,380,831; 5,436,391; and Murray et al. (1989) Nucleic Acids Res 17:477-498. Additional sequence modifications can enhance gene expression in a cellular host including elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. The sequence may also be modified to avoid predicted hairpin secondary mRNA structures.

Compositions and methods for inhibiting or eliminating the expression of a gene in a plant are well known. Reduction of the activity of specific genes may be desirable for several aspects of genetic engineering in plants. Many techniques for gene silencing are known, including but not limited to antisense technology (see, e.g., Sheehy et al. (1988) Proc Natl Acad Sci USA 85:8805-8809; and U.S. Pat. Nos. 5,107,065; 5,453, 566; and 5,759,829); cosuppression (e.g., Taylor (1997) Plant Cell 9:1245; Jorgensen (1990) Trends Biotech 8:340-344; Flavell (1994) Proc Natl Acad Sci USA 91:3490-3496; Finnegan et al. (1994) Bio/Technology 12:883-888; and Neuhuber et al. (1994) Mol Gen Genet 244:230-241); RNA interference (Napoli et al. (1990) Plant Cell 2:279-289; U.S. Pat. No. 5,034,323; Sharp (1999) Genes Dev 13:139-141; Zamore et al. (2000) Cell 101:25-33; Javier (2003) Nature 425:257-263; and, Montgomery et al. (1998) Proc Natl Acad Sci USA 95:15502-15507), virus-induced gene silencing (Burton et al. (2000) Plant Cell 12:691-705; and Baulcombe (1999) Curr Op Plant Bio 2:109-113); target-RNA-specific ribozymes (Haseloff et al. (1988) Nature 334: 585-591); hairpin structures (Smith et al. (2000) Nature 407:319-320; WO99/53050; WO02/00904; and WO98/53083); ribozymes (Steinecke et al. (1992) EMBO J 11:1525; U.S. Pat. No. 4,987,071; and, Perriman et al. (1993) Antisense Res Dev 3:253); oligonucleotide mediated targeted modification (e.g., WO03/076574: and WO99/25853); Zn-finger targeted molecules (e.g., WO01/52620; WO03/048345; and WO00/42219); microRNA (miRNA) and/or siRNAs (e.g., US2005/0138689; and US2005/0120415); and other methods, or combinations of the above methods.

Transformation includes any method to deliver the composition of interest to the interior of a cell, encompassing transient and stable transformation of cells with a polynucleotide composition, transient delivery of a polypeptide composition, and transient and stable delivery of a microorganism, and the like. Transient transformation is the delivery of a composition of interest to the interior of a cell wherein the composition of interest is not stably inherited by progeny of that cell. Stable transformation is the delivery of a composition of interest to the interior of a cell wherein the composition of interest is stably inherited by progeny of that cell. Transformation frequency is a measure of the number of cells to which the composition of interest has been delivered, and can be measured by a number of standard assays including but not limited to, transient expression of a polynucleotide of interest, transient presence of a polypeptide or other composition of interest, and frequency of stable integration of a polynucleotide of interest into a genome. Transformation protocols as well as protocols for introducing polypeptides or polynucleotide sequences into plants may vary depending on the type of plant or plant cell targeted for transformation. Suitable methods of introducing polypeptides and polynucleotides into plant cells include microinjection (Crossway et al. (1986) Biotechniques 4:320-334, U.S. Pat. No. 6,300,543), electroporation (Riggs et al. (1986) Proc Natl Acad Sci USA 83:5602-5606, Agrobacterium-mediated transformation (U.S. Pat. Nos. 5,563,055; and 5,981,840), direct gene transfer (Paszkowski et al. (1984) EMBO J 3:2717-2722), and ballistic particle acceleration (U.S. Pat. Nos. 4,945,050; 5,879,918; 5,886,244; and 5,932,782; Tomes et al. (1995) in Plant Cell, Tissue, and Organ Culture: Fundamental Methods, ed. Gamborg & Phillips (Springer-Verlag, Berlin); McCabe et al. (1988) Biotechnology 6:923-926); Weissinger et al. (1988) Ann Rev Genet 22:421-477; Sanford et al. (1987) Particulate Science and Technology 5:27-37 (onion); Christou et al. (1988) Plant Physiol 87:671-674 (soybean); Finer & McMullen (1991) In Vitro Cell Dev Biol 27P:175-182 (soybean); Singh et al. (1998) Theor Appl Genet 96:319-324 (soybean); Datta et al. (1990) Biotechnology 8:736-740 (rice); Klein et al. (1988) Proc Natl Acad Sci USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol 91:440-444 (maize); Fromm et al. (1990) Biotechnology 8:833-839 (maize); Hooykaas-Van Slogteren et al. (1984) Nature 311:763-764; U.S. Pat. No. 5,736,369 (cereals); Bytebier et al. (1987) Proc Natl Acad Sci USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Rep 9:415-418; Kaeppler et al. (1992) Theor Appl Genet 84:560-566; Frame et al. (1994) Plant J 6941-948; and Brisibe et al. (2000) J Exp Bot 51:187-196 (whiskers); Li et al. (1993) Plant Cell Rep 12:250-255; Christou & Ford (1995) Ann Bot 75:407-413 (rice); and Ch. 8, pp. 189-253 in Advances in Cellular and Molecular Biology of Plants, Vol. 5, Ed. Vasil, Kluwer Acad Publ (Dordrecht, The Netherlands) 1999. Direct delivery methods using microparticles are well established for a wide variety of plant species and tissues, any method can be used with the compositions and methods provided herein.

A polynucleotide of interest may be introduced into plants by contacting plants with a virus or viral nucleic acid. Generally, such methods involve incorporating a desired polynucleotide within a viral DNA or RNA molecule. The sequence may initially be synthesized in a viral polyprotein and later processed in vivo or in vitro to produce a desired protein. Useful promoters encompass promoters utilized for transcription by viral RNA polymerases. Methods for introducing polynucleotides into plants and expressing a protein encoded, involving viral DNA or RNA molecules, are known, see, e.g., U.S. Pat. Nos. 5,889,191; 5,889,190; 5,866,785; 5,589,367; 5,316,931; and Porta et al. (1996) Mol Biotech 5:209-221. Viral strains, and their viral nucleic acids, include, but are not limited to, geminivirus, begomovirus, curtovirus, mastrevirus, (−) strand RNA viruses, (+) strand RNA viruses, potyvirus, potexvirus, tobamovirus, or other DNA viruses, nanoviruses, viroids, and the like, for example, African cassava mosaic virus (ACMV) (Ward et al. (1988) EMBO J 7:899-904 and Hayes et al. (1988) Nature 334:179-182), barley stripe mosaic virus (BSM) (Joshi et al. (1990) EMBO J 9:2663-2669), cauliflower mosaic virus (CaMV) (Gronenborn et al. (1981) Nature 294:773-776 and Brisson et al. (1984) Nature 310:511-514), maize streak virus (MSV) (Lazarowitz et al. (1989) EMBO J 8:1023-1032 and Shen et al. (1994) J Gen Virol 76:965-969), tobacco mosaic virus (TMV) (Takamatsu et al. (1987) EMBO J 6:307-311 and Dawson et al. (1989) Virology 172:285-292), tomato golden mosaic virus (TGMV) (Elmer et al. (1990) Nucleic Acids Res 18:2001-2006), and wheat dwarf virus (WDV) (Woolston et al. (1989) Nucleic Acids Res 17:6029-6041) and derivatives thereof. See also, Porat et al. (1996) Mol Biotechnol 5:209-221.

A variety of bacterial strains may be introduced into a plant. In some examples an Agrobacterium comprising a T-DNA containing a polynucleotide of interest is provided to a plant cell by direct delivery via microparticles, wherein the Agrobacterium is capable of T-DNA transfer into a plant cell. A number of wild-type and disarmed strains of Agrobacterium tumefaciens and Agrobacterium rhizogenes harboring T-DNA, Ti or Ri plasmids can be used. Reviews of Agrobacterium-mediated transformation in monocots and dicots include for example, Hellens et al. (2000) Trends Plant Sci 5:446-451; Hooykaas (1989) Plant Mol Biol 13:327-336; Smith et al. (1995) Crop Sci 35:301-309; Chilton (1993) Proc Natl Acad Sci USA 90:3119-3210; and Moloney et al. (1993) In: Monograph TheorAppl Genet, N.Y., Springer Verlag 19:148-167. Agrobacterium can be provided directly to cells by particle bombardment as described in U.S. Pat. No. 5,932,782, or co-incubation with bombardment-wounded cells as described in EP 0486233, both of which are herein incorporated by reference. Agrobacterium strains of interest can be wild type or derivatives thereof which have alterations that increase transformation efficiency. Strains of interest include, but are not limited to, A. tumefaciens strain C58, a nopaline-type strain (Deblaere et al. (1985) Nucleic Acids Res 13:4777-4788); octopine-type strains such as LBA4404 (Hoekema et al. (1983) Nature 303:179-180); or succinamopine-type strains e.g., EHA101 or EHA105 (Hood et al. (1986) J Bacteriol 168:1291-1301); A. tumefaciens strain A281 (U.S. Patent Publication No. 20020178463); GV2260 (McBride et al. (1990) Plant Mol Biol 14:269-276); GV3100 and GV3101 (Holsters et al. (1980) Plasmid 3:212-230); A136 (Watson et al. (1975) J Bacteriol 123:255-264); GV3850 (Zambryski et al. (1983) EMBO J 2:2143-2150); GV311::Pmp90 (Koncz et al. (1986) Mol Gen Genet 204:383-396); and, AGL-1 (Lazo et al. (1991) Biotechnology 9:963-967).

Transfer DNA or T-DNA comprises a genetic element that is capable of integrating a polynucleotide contained within its borders into another polynucleotide. The T-DNA can comprise the entire T-DNA, but need only comprise the minimal sequence necessary for cis transfer, typically the right or left border is sufficient. The T-DNA can be synthetically derived or can be from an A. rhizogene Ri plasmid or from an A. tumefaciens Ti plasmid, or functional derivatives thereof. Any polynucleotide to be transferred, for example a recombinase, a polynucleotide of interest, a recombination site, a restriction site, a recognition site, a sequence tag, a target site, a transfer cassette and/or a marker sequence may be positioned between the left border sequence and the right border sequence of the T-DNA. The sequences of the left and right border sequences may or may not be identical and may or may not be inverted repeats of one another. It is also possible to use only one border, or more than two borders, to accomplish transfer of a desired polynucleotide. Various plasmids are available comprising T-DNAs that can be employed in the methods. For example, many Agrobacterium employed for the transformation of dicotyledonous plant cells contain a vector having a DNA region originating from the virulence (vir) region of the Ti plasmid. The Ti plasmid originated from A. tumefaciens, and the polynucleotide of interest can be inserted into this vector. Alternatively, the polynucleotide of interest can be contained in a separate plasmid which is then inserted into the Ti plasmid in vivo, in Agrobacterium, by homologous recombination or other equivalently resulting processes. See, for example, Herrera-Esterella et al. (1983) EMBO J 2:987-995 and Horch et al. (1984) Science 223:496-498. Also available is a vector containing a DNA region originating from the virulence (vir) region of Ti plasmid pTiBo542 (Jin et al. (1987) J Bacteriol 169:4417-4425) contained in a super-virulent A. tumefaciens strain A281 and showing extremely high transformation efficiency. This vector includes regions that permit vector replication in both E. coli and Agrobacterium, is known as a superbinary vector (see European Patent Application 0604662A1). See, Hood et al. (1984) Bio/Tech 2:702-709; Komari et al. (1986) Bacteriol 166:88-94. Examples of superbinary vectors include pTOK162 and pTIBo542 (US2002/178463 and Japanese Laid-Open Patent Application No. 4-222527); pTOK23 (Komari et al. (1990) Plant Cell Rep 9:303-306); pPHP10525 (U.S. Pat. No. 6,822,144), see, also Ishida et al. (1996) Nat Biotech 14:745-750. Additional transformation vectors comprising T-DNAs that can be used further include, but are not limited to, pBIN19 (Bevan et al. (1984) Nucleic Acids Res 12:8711-8721); pC22 (Simoens et al. (1986) Nucleic Acids Res 14:8073-8090); pGA482 (An et al. (1985) EMBO J 4:277-284); pPCV001 (Koncz et al. (1986) Mol Gen Genet 204:383-396); pCGN1547 (McBride et al. (1990) Plant Mol Biol 14:269-276); pJJ1881 (Jones et al. (1992) Transgenic Res 1:285-297); pPzP111 (Hajukiewicz et al. (1994) Plant Mol Biol 25:989-994); and, pGreen0029 (Hellens et al. (2000) Plant Mol Biol 42:819-832).

The term plant includes plant cells, plant protoplasts, plant cell tissue cultures from which a plant can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Progeny, variants, and mutants of the regenerated plants are also included. Any plant species can be used with the methods and compositions, including, but not limited to, monocots and dicots. Examples of plant genera and species include, but are not limited to, maize (Zea mays), Brassica sp. (e.g., B. napus, B. rapa, B. juncea), castor, palm, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), Arabidopsis thaliana, oats (Avena spp.), barley (Hordeum spp.), leguminous plants such as guar beans, locust bean, fenugreek, garden beans, cowpea, mungbean, fava bean, lentils, and chickpea, vegetables, ornamentals, grasses and conifers. Vegetables include tomatoes (Lycopersicon esculentum), lettuce (e.g., Lactuca sativa), green beans (Phaseolus vulgaris), lima beans (Phaseolus limensis), peas (Pisium spp., Lathyrus spp.), and Cucumis species such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers include pines, for example, loblolly pine (Pinus taeda), slash pine (Pinus elliotii), ponderosa pine (Pinus ponderosa), lodgepole pine (Pinus contorta), and Monterey pine (Pinus radiata), Douglas fir (Pseudotsuga menziesii); Western hemlock (Tsuga canadensis), Sitka spruce (Picea glauca), redwood (Sequoia sempervirens), true firs such as silver fir (Abies amabilis) and balsam fir (Abies balsamea), and cedars such as Western red cedar (Thuja plicata) and Alaska yellow cedar (Chamaecyparis nootkatensis).

Plant callus, explants, organs, or parts thereof can be regenerated to form plants. Regeneration techniques are described generally in Klee et al. Ann Rev Plant Phys (1987) 38:467-486. The cells produced by the methods may be grown into plants using standard techniques and media (e.g., McCormick et al. (1986) Plant Cell Rep 5:81-84; Gruber et al. (1993) Vectors for Plant Transformation, In: Methods in Plant Molecular Biology and Biotechnology; Glick & Thompson, Eds., CRC Press, Inc., Boca Raton, pages 89-119; Gordon-Kamm et al. (1990) Plant Cell 2:603-618). These plants may then be grown and self-pollinated, backcrossed, and/or outcrossed, and the resulting progeny having the desired characteristic(s) identified. Two or more generations may be grown to ensure that the characteristic is stably maintained and inherited and then seeds harvested. In this manner transformed/transgenic seed having a recited a construct stably incorporated into their genome are provided. A plant and/or a seed having stably incorporated the construct can be further characterized for expression, site-specific integration potential, agronomics, and copy number (see, e.g., U.S. Pat. No. 6,187,994).

### EXAMPLE 1

A. Compounds and Preparation of Microparticles

A variety of compounds can be tested for their ability to associate a composition of interest with a microparticle to provide a means to directly deliver the composition of interest to a cell.

i. The cationic liposome, Lipofectin® (InVitrogen, Carlsbad, Calif., USA), can be used to associate nucleic acids to microparticles. These DNA coated microparticles can then be delivered to maize cells, using particle bombardment where marker genes are expressed in the host plant cells.

In this example, plasmid DNA (PHP3957) harboring the beta-glucuronidase (GUS) marker gene was associated to 1 μm tungsten particles and bombarded into immature maize embryos. Three days after bombardment, transient expression of GUS was visualized using standard histochemical staining.

Prior to associating the DNA with the particles, the Lipofectin® is diluted 1:1 with sterile distilled water just prior to use. Next, 15 μl of plasmid DNA (0.05 μg/ml) was mixed with 15 μL of diluted Lipofectin®. The DNA and Lipofection® was mixed gently by hand. The DNA:Lipofectin® mixture was added to a tube containing 1 μM tungsten particles and mixed gently by hand until evenly dispersed. Immediately after mixing the DNA:Lipofection®:particle mixture. 10 μL was added to individual macrocarriers that are used for standard helium gun particle bombardment. The macrocarriers containing DNA:Lipofection®:particle mixture were placed on a warm heating plate until the mixture was just dry. Once the DNA:Lipofection®:particle mixture dried, the coated macrocarriers were used to deliver the DNA immature maize embryos using standard particle bombardment procedures.

Bombarded embryos were incubated at 280 C in the dark for 3 days on an N6 media containing 20% sucrose, 2,4-D, vitamins and gelrite as the gelling agent. The embryos were then incubated with standard buffer containing X-Gluc to visualize activity of the GUS enzyme. Strong transient expression of the delivered GUS gene was observed in the embryos that were bombarded using this procedure.

ii. The cationic lipid TFX-50™ (Promega, Madison, Wis. USA), histone HI (Sigma Chemical Co., St. Louis, Mo., USA), and PEI were also tested for their ability to associate a polynucleotide composition or a protein-polynucleotide composition with a microparticle for particle bombardment. All compounds tested were capable of delivering the composition of interest to a cell.

B. Uniformity of Microparticles and Microparticle Suspension

Sample of microparticles were examined by microscopy to determine the level of aggregation and uniformity of coating for untreated, control, and polynucleotide preparations. Aggregation of microparticles reduces the ease of resuspending the particles, and the uniformity of suspension. Extreme clumping may result in more physical force or extreme conditions being used to resuspend prepared particles, possibly damaged the attached composition of interest, for example shearing of DNA.

i. Tungsten 1.8 μm Particles

As observed using scanning electron microscopy, naked tungsten particles, 1.8 μm avg, form small aggregates in the absence of salts and DNA. When treated with CaCl₂-spermidine, aggregates at least as large, typically larger than the naked particles are still observed, but no salt deposits are observed in the aggregates. However, tungsten particles treated with DNA-CaCl₂-spermidine showed a high degree of aggregation, with sticky salt deposits or “bridges” were observed on and between some particles in the aggregates. The level of these deposits varied. Particles treated with DNA-CaCl₂-spermidine were also examined by transmission electron microscopy after negative staining and atomic force microscopy both examinations showed a non-uniform, spotty distribution of the DNA on the particle.

ii. Gold 1.0 μm Particles

As observed using scanning electron microscopy, naked gold particles, 1.0 μm avg, do not aggregate in the absence of salts and DNA, but remain as single particles. Gold particles treated with DNA-TFX-50™ showed very little aggregation of the particles, with the particles generally remaining as single particles. Any aggregates observed were typically composed of only a few particles, approximately 10 or fewer aggregated particles. No deposits or “bridges” were observed in the DNA-TFX-50™ gold particle aggregates examined.

### EXAMPLE 2

Microparticles can be prepared to deliver any polynucleotide(s), oligonucleotide(s), polypeptide(s), other compound(s), molecules, and/or microorganism(s), or any combination thereof to plants, plant cells, and/or plant tissues. Microparticle-based transformation methods are well known and any such method may be used to deliver the prepared particles.

A. Delivery of Cationic Oligonucleotides

Cationic oligonucleotides can be delivered to plant cells on biolistic particles prepared using a cationic lipid, such as TFX-50. Cationic oligonucleotides can be used for targeted modification of a DNA sequence in the genome, see for example U.S. patent publication 2004/0023262 herein incorporated by reference. In one example, plant cells comprising PHP1 1207 Ubi pro::moPAT::TAG::GFP::pinil were used as the target for cationic oligonucleotides designed to convert the stop codon TAG to TAC (tyrosine) to allow expression of GFP. Four oligonucleotides, as well as controls, were introduced into the callus tissue and/or 10DAP embryos of four different GFP target lines using a biolistic gun method. For microprojectile bombardment gold particles (60 μg/μl) were prepared with the cationic oligonucleotides or plasmid controls (0.1 μg/μl) and TFX-50 (5 μl of TFX-50 for 1.0 μg of DNA) then resuspended in 100% ethanol. As shown in Table 1, treatments A-D used different cationic oligos for delivery into the target lines. Treatment E is a negative control, where neither a plasmid nor an oligo was bombarded into GS3 callus. Treatment F is positive control PHP7921 contains Ubi pro::GFP to evaluate particle and/or oligo delivery. All PHP7921 events showed some GFP spots. Treatment G used callus from a stably transformed line of PHP1 7228 Ubi pro::moPAT::GFP without the TAG stop codon to compare GFP expression in PHP17228 callus material versus induced GFP activation under similar biolistic /culture conditions. PHP17228 material was GFP positive. The bombarded plates containing callus cultures or embryos were screened using Leica DC200 microscope with a GFP2 filter at least twice between day 1 and 10 after bombardment.

Preliminary results indicated that cationic oligos complementary to either transcribed or non-transcribed DNA strand were capable of making the correction that activates GFP. The results indicated that particles treated with either oligonucleotides or plasmids effectively delivered the polynucleotides to the target cells.

TABLE 1

| | Oligo/Plasmid | | Sel | | |
|---|---|---|---|---|---|
| Treatment | (0.1 ug/ul) | Loading | media | Pressure | Description |
| A | GMOPHP12 | TFX-50 | 560R | 650 psi | Experiment |
| B | GMOPHP13 | TFX-50 | 560R | 650 psi | Experiment |
| C | GMOPHP17 | TFX-50 | 560R | 650 psi | Experiment |
| D | GMOPHP18 | TFX-50 | 560R | 650 psi | Experiment |
| E | none | TFX-50 | 560R | 650 psi | Control |
| F | PHP7921 | TFX-50 | 560R | 650 psi | Control |
| G | none | TFX-50 | 560R | 650 psi | Control |

B. Delivery of Recombination Substrate and Transient Protein Expression

Immature embryos from a corn line having a site-specific recombination target site comprising two non-identical recombination sites can be used for subsequent re-transformation with a transfer cassette comprising a polynucleotide of interest flanked by the two non-identical recombination sites using standard particle bombardment methods. The target sites, transfer cassettes, and target lines are created essentially as described in WO 99/25821, herein incorporated by reference. Plasmids comprising the transfer cassette are co-transformed into immature embryos from their respective target lines along with plasmid PHP5096 (Ubi:Ubi-intron::FLPm::pinII). The transfer cassette plasmid is mixed with the FLP-containing plasmid (PHP5096), using 100 ng of the FRT-containing transfer cassette plasmid and 10 ng of the FLP plasmid per bombardment. The FLP plasmid provides transient expression of FLP recombinase without integration of the polynucleotide into the genome of the target cells.

To prepare DNA for delivery, DNA solutions are added to 50 μl of a gold-particle stock solution (0.1 μg/μl of 0.6 micron gold particles). For example, 10 μl of a 0.1 μg/μl solution of transfer cassette plasmid, and 10 μl of a 0.01 μg/μl solution of PHP5096 are first added to 30 μl of water. To this DNA mixture, 50 μl of the gold stock solution is added and the mixture briefly sonicated. Next 5 μl of TFX-50 (Promega Corp., 2800 Woods Hollow Road, Madison Wis. 53711) is added and the mixture is placed on a rotary shaker at 100 rpm for 10 minutes. The mixture is briefly centrifuged to pellet the gold particles and remove supernatant. After removal of the excess DNA/TFX solution, 120 μl of absolute EtOH is added, and 10 μl aliquots are dispensed onto the macrocarriers typically used with the DuPont PDS-1000 Helium Particle Gun. The gold particles with adhered DNA are allowed to dry onto the carriers and then these are used for standard particle bombardment. After re-transformation the immature embryos are placed onto 560P medium for two weeks to recover, and then moved to selection medium, to identify re-transformation events which are subsequently regenerated into plants using standard methods.

C. Delivery of Large Polynucleotides and Polynucleotide Mixtures

Polynucleotide compositions comprising at least one large polynucleotide greater than about 20 kb can be delivered by preparing microparticles as described herein. In this example, large polynucleotide constructs and/or mixtures of large constructs derived from BAC clones are delivered, with the constructs ranging from 20 kb to over 200 kb in size which can be provided as a circular plasmid or in a linearize form, see for example U.S. Provisional Ser. No. 60/801,004 filed May. 18, 2006, herein incorporated by reference.

Immature maize embryos from 8-11 DAP are surface sterilized in a solution of 30% bleach plus 0.5% Micro detergent for 20 minutes, and rinsed two times with sterile water. The immature embryos are excised, placed embryo axis side down (scutellum side up), 50 embryos per plate, on 560L medium for 1-3 days at 26° C. in the dark. Before transformation the immature embryos are transferred on to 560Y medium for 4 hours, and then aligned within the 2.5-cm target zone in preparation for bombardment.

The DNA is adhered onto 0.6 μm (average diameter) gold pellets using a water-soluble cationic lipid Tfx™-50 (Cat# E1811, Promega, Madison, Wis., USA) as follows: prepare DNA solution on ice using 1 μg of maize centromeric BAC DNA construct (10 μl ); optionally other constructs for co-bombardment such as 50 ng (0.5 μl ) PHP21875 (BBM), and 50 ng (0.5 μl ) PHP21139 (WUS); mix DNA solution. To the pre-mixed DNA add 20 μl prepared gold particles (15 mg/ml) in water; 10 μl Tfx-50 in water; mix carefully. This can be stored on ice during preparation of macrocarriers, typically about 10 min. Pellet gold particles in a microfuge at 10,000 rpm for 1 min, remove supernatant. Carefully rinse the pellet with 100 ml of 100% EtOH without resuspending the pellet, carefully remove the EtOH rinse. Add 20 μl of 100% EtOH and carefully resuspend the particles by brief sonication, 10 μl spotted onto the center of each macrocarrier and allowed to dry about 2 minutes before bombardment. The sample plates of maize target embryos are bombarded twice per plate using approximately 0.5 μg of DNA per shot using the Bio-Rad PDS-1000 He device (Bio-Rad Laboratories, Hercules, Calif.) with a rupture pressure of 450 PSI, a vacuum pressure of 27-28 inches of Hg, and a particle flight distance of 8.5 cm.

D. Delivery of Agrobacterium

Microorganisms, such as bacteria, bacteria comprising a bacteriophage, or a virus can be delivered via direct delivery of microparticles comprising the microorganism. For example, Agrobacterium comprising a T-DNA which comprises a polynucleotide of interest have been adhered to microparticles for delivery to plant cell by particle bombardment, producing a fertile transgenic plant comprising the polynucleotide of interest stably incorporated in its genome, see for example U.S. Pat. No. 5,932,782, herein incorporated by reference.

In Bidney (U.S. Pat. No. 5,932,782), Agrobacterium comprising a T-DNA were grown in standard media to various cell densities, mixed with gold particles, applied to macroprojectiles, and dried for varying times. These preparations were used to bombard plant tissues.

Various association agents can be tested for their effect on microorganism growth and/or viability, concentration, temperature of preparation, appropriate microorganism cell density, plant cell transformation frequency and/or stability. For example, Agrobacterium cultures comprising a T-DNA with a polynucleotide of interest, such as a visible marker and/or selectable marker, can be growth in standard media to densities of 0.5-2.0 OD₆₀₀, and aliquots of the cultures mixed with varying concentrations of TFX-50. This is mixed with a fixed quantity of gold particles, and aliquots applied to macrocarriers and dried for varying amounts of time. The macrocarriers are used to bombard a plant tissue, such as immature maize embryos prepared using standard conditions. Various microparticle compositions, as well as microparticle sizes can be tested as well. Bombarded tissue is regenerated under standard conditions and screened for the polynucleotide(s) of interest. Bacterial media plates can be bombarded to determine Agrobacterium viability under the experimental conditions.

Although the foregoing invention has been described in some detail by way of illustration and example for purposes of clarity of understanding, certain changes, modifications, and derivations may be practiced within the scope of the appended claims.

The article “a” and “an” are used herein to refer to one or more than one (i.e., to at least one) of the grammatical object of the article. By way of example, “an element” means one or more than one element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

What is claimed is:

1. A method to prepare microparticles for direct delivery of a composition of interest to plant cells comprising:
a) providing microparticles suitable for direct-delivery into plant cells;
b) providing the composition of interest; and,
c) contacting the composition of interest with the microparticles in the presence of a compound to produce a microparticle having the composition of interest attached thereto, wherein the compound is selected from the group consisting of a cationic lipid solution, a liposome solution, a cationic polymer, a cationic protein, a cationic peptide, and a cationic polyamino acid.

2. The method of claim 1 wherein the microparticles are selected from the group consisting of gold particles, tungsten particles, and silicon carbide whisker particles.

3. The method of claim 1 wherein the compound is a cationic lipid solution comprising N,N,N′,N′-tetramethyl-N,N′-bis(2-hydroxylethyl)-2,3-di(oleoyloxy)-1,4-butanediammonium iodide.

4. The method of claim 3, wherein the cationic lipid solution further comprises L-dioleoyl phosphatidylethanolamine (DOPE).

5. The method of claim 1 wherein the compound is selected from the group consisting of Tfx-10™, Tfx-20™, Tfx-50™, Lipofectin™, Lipofectamine™, Cellfectin™, Effectene™, Cytofectin GSV™, Perfect Lipids™, DOTAP™, DMRIE-C™, FuGENE-6™, Superfect™, Polyfect™, polyethyleneimine, chitosan, protamine CI, histone H1, histone CENH3, poly-L lysine, and DMSA.

6. The method of claim 1 wherein the composition of interest comprises a composition selected from the group consisting of a polynucleotide composition, a polypeptide composition, a subcellular organelle composition, and a microorganism composition.

7. The method of claim 6 wherein the composition is a polynucleotide composition comprising at least one polynucleotide encoding a polypeptide that enhances or stimulates cell growth, a recombinase, an integrase, a site-specific recombinase, a homing meganuclease, a transposase, a meganuclease, a restriction enzyme, a transcription factor, a repressor, a screenable marker, and/or a zinc-finger protein.

8. The method of claim 7 wherein the polynucleotide composition comprises a polynucleotide encoding a polypeptide that enhances or stimulates cell growth, wherein the polypeptide is selected from the group consisting of a wuschel polypeptide and a babyboom polypeptide.

9. The method of claim 6 wherein the composition is a polynucleotide composition comprising a mixture of polynucleotide species.

10. The method of claim 6 wherein the composition is a polynucleotide composition comprising at least one polynucleotide greater than 20 kb in size.

11. A microparticle produced by the method of claim 1, wherein the microparticle has the composition of interest attached thereto.

12. A direct-delivery composition comprising microparticles, a composition of interest, and a compound selected from the group consisting of a cationic lipid solution, a liposome solution, a cationic polymer, a cationic protein, a cationic peptide, and a cationic polyamino acid.

13. A method to provide a composition of interest to plant cells comprising:
a) providing microparticles suitable for direct delivery into plant cells;
b) providing the composition of interest;
c) contacting the composition of interest with the microparticles in the presence of a compound to produce microparticles having the composition of interest attached thereto, wherein the compound is selected from the group consisting of a cationic lipid solution, a liposome solution, a cationic polymer, a cationic protein, a cationic peptide, and a cationic polyamino acid; and,
d) contacting the microparticles produced in step (c) with plant cells such that the microparticles deliver the composition of interest to the interior of the plant cells.

14. The method of claim 13, wherein the microparticles are selected from the group consisting of gold particles, tungsten particles, and silica carbide whisker particles.

15. The method of claim 13, wherein the compound is a cationic lipid solution comprising N,N,N′,N′-tetramethyl-N,N′-bis(2-hydroxylethyl)-2,3-di(oleoyloxy)-1,4-butanediammonium iodide.

16. The method of claim 15, wherein the cationic lipid solution further comprises L-dioleoyl phosphatidylethanolamine (DOPE).

17. The method of claim 13 wherein the compound is selected from the group consisting of Tfx-10™, Tfx-20™, Tfx-50™, Lipofectin™, Lipofectamine™, Cellfectin™, Effectene™, Cytofectin GSV™, Perfect Lipids™, DOTAP™, DMRIE-C™, FuGENE-6™, Superfect™, Polyfect™, polyethyleneimine, chitosan, protamine CI, histone H1, histone CENH3, poly-L lysine, and DMSA.

18. The method of claim 13 wherein the composition of interest comprises a composition selected from the group consisting of a polynucleotide composition, a polypeptide composition, a subcellular organelle composition, and a microorganism composition.

19. The method of claim 13 wherein the composition is a polynucleotide composition comprising at least one polynucleotide encoding a polypeptide that enhances or stimulates cell growth, a recombinase, an integrase, a site-specific recombinase, a homing meganuclease, a transposase, a meganuclease, a restriction enzyme, a transcription factor, a repressor, a screenable marker, and/or a zinc-finger protein.

20. The method of claim 19 wherein the polynucleotide composition comprises a polynucleotide encoding a polypeptide that enhances or stimulates cell growth, wherein the polypeptide is selected from the group consisting of a wuschel polypeptide and a babyboom polypeptide.

21. The method of claim 19 wherein the composition is a polynucleotide composition comprising a mixture of polynucleotide species.

22. The method of claim 19 wherein the composition is a polynucleotide composition comprising at least one polynucleotide greater than 20 kb in size.

23. The method of claim 13 wherein the plant cell is from a monocotyledonous or a dicotyledonous plant.

24. The method of claim 23, wherein the plant cell is selected from the group consisting of maize, rice, wheat, barley, millet, sorghum, rye, soybean, alfalfa, canola, Arabidopsis, tobacco, sunflower, cotton, and safflower.

25. The method of claim 13 wherein after delivery of the microparticles into plant cells the composition of interest does not substantially dissociate from the microparticles.

26. The method of claim 13 wherein the composition of interest comprises a polynucleotide composition comprising at least one polynucleotide of interest, wherein after delivery of the microparticles the polynucleotide of interest stably incorporates into a genome of a plant cell.

27. The method of claim 26, wherein polynucleotide of interest incorporates into the genome by means of a site-specific recombinase mediated recombination event.

28. The method of claim 13, wherein the composition of interest comprises a polynucleotide composition comprising at least one polynucleotide of interest, wherein after delivery of the microparticles the polynucleotide of interest does not stably incorporate into a genome of a plant cell.

29. The method of claim 28, wherein after delivery of the microparticles the polynucleotide of interest is transiently expressed in the plant cells.

30. The method of claim 13 wherein the composition of interest comprises a polynucleotide composition comprising a polynucleotide of interest, further comprising recovering a plant cell comprising the polynucleotide interest.

31. The method of claim 30, wherein the polynucleotide of interest is incorporated into a genome of the plant cell to produce a stably transformed plant cell.

32. The method of claim 31 further comprising recovering a plant comprising the polynucleotide interest stably incorporated into a genome of the plant.

33. The method of claim 32 further comprising recovering a seed comprising the polynucleotide interest stably incorporated into a genome of the seed.

34. The method of claim 30 wherein the frequency of recovering the plant cell comprising the polynucleotide of interest is increased as compared to a control, wherein the control comprises contacting the polynucleotide of interest with the microparticles using a standard calcium chloride precipitation method.

35. The method of claim 34, wherein the frequency of recovering the plant cell comprising the polynucleotide of interest is increased at least about 1.5 fold.

36. The method of claim 34 wherein the polynucleotide of interest is incorporated into a genome of the plant cell to produce a stably transformed plant cell.
